(19) United States
(12) Patent Application Publication
(10) Pub. No.: US 2026/0207853 A1
(43) Pub. Date: Jul. 23, 2026

(54) Medicament delivery device

(71) Applicant: SHL Medical AG, Zug (CH)

(72) Inventor: Nurettin Ali, Deerfield Beach, FL (US)

(21) Appl. No.: 19/143,030

(22) PCT Filed: Dec. 22, 2023
(86) PCT No.: PCT/EP2023/087587

§ 371 Date: Jun. 25, 2025

Related U.S. Application Data
(60) Provisional application No. 63/436,993, filed on Jan. 4, 2023.

(30) Foreign Application Priority Data
Feb. 2, 2023 (EP) 23154619.3

(51) Int. Cl. A61M 5/32; A61M 5/20

(52) U.S. Cl. CPC A61M 5/3202; A61M 5/2033; A61M 2005/2073

(57) ABSTRACT

The present disclosure provides a medicament delivery device having a proximal end pointing towards a dose delivery site during use of the medicament delivery device, and a distal end opposite the proximal end. The medicament delivery device includes a housing having a longitudinal axis, and a medicament delivery member cover axially moveable with respect to the housing. The housing comprises an inner cylindrical portion, the cylindrical portion comprising a flange extending in the radial outward direction with respect to the longitudinal axis. The medicament delivery member cover includes at least one medicament delivery member cover arm extending in a distal direction away from a medicament delivery site. The medicament delivery member cover arm comprises a flexible locking arm extending in a radial inward direction. The flexible locking arm is configured to abut the flange and to block proximal movement of the medicament delivery member cover towards the medicament delivery site.

### TECHNICAL FIELD

The present disclosure relates to a medicament delivery device and in particular to an assembly of a medicament delivery device.

### BACKGROUND

A medicament delivery device, in particular an autoinjector, according to the prior art comprises a housing comprising a front housing and a rear housing. The housing is adapted to hold a medicament container, such as a syringe.

The syringe may be a pre-filled syringe and have a needle as a medicament delivery member arranged at a proximal end. When the autoinjector and/or the syringe are assembled, a protective needle sheath may be removably coupled to the needle. The protective needle sheath may be made of a flexible shell (for instance composed of rubber) and/or a rigid shell (for instance composed of plastic). A stopper, also called plunger, may be arranged for sealing the syringe distally and for expelling a medicament contained in the syringe through the needle. In other exemplary embodiments, the medicament container may be a cartridge which includes the medicament and engages a removable needle (e.g., by threads, snaps, friction, etc.).

A cap may be removably disposed at a proximal end of the housing. The cap may include an element (e.g., a barb, a hook, a narrowed section, etc.) arranged to engage the protective needle sheath, the housing and/or a medicament delivery member cover, also referred to as needle cover, telescoped within the housing. The cap may comprise grip features for facilitating removal of the cap (e.g., by twisting and/or pulling the cap relative to the housing).

A medicament delivery member cover biasing member (for instance a coil spring) may be arranged to bias the medicament delivery member cover in a proximal direction relative to the housing, before injection, for safety reasons. The medicament delivery member cover biasing member needs to be compressed at the beginning of injection in order to retract the medicament delivery member cover inside the housing and uncover the medicament delivery member, thereby activating the device.

A pre-loaded drive biasing member, for instance a pre-compressed drive coil spring, may be arranged within the housing. A plunger rod may be used to forward force from the drive biasing member to the stopper. In an exemplary embodiment, the plunger rod is hollow and the drive biasing member is arranged within the plunger rod. In another exemplary embodiment, the plunger rod is solid and the drive biasing member engages a distal end of the plunger rod. When the pre-loaded drive biasing member is released, the drive biasing member biases the plunger rod in the proximal direction relative to the housing, releasing the plunger rod. Besides that, the plunger rod may itself comprise means for sealing the medicament container such that no stopper needs to be provided.

A plunger rod release mechanism may be arranged for preventing release of the plunger rod prior to retraction of the medicament delivery member cover relative to the housing and for releasing the plunger rod only once the medicament delivery member cover is sufficiently retracted.

A medicament delivery member cover lock mechanism (needle cover lock mechanism) may be arranged to prevent retraction of the medicament delivery member cover relative to the housing when the cap is in place, thereby avoiding unintentional activation of the autoinjector (e.g., if dropped, during shipping or packaging, etc.). Such mechanisms are known in the art and will not be described in detail.

Due to the number of parts necessary for a medicament delivery device and the complexity of their interaction, an assembly process of such devices is often complicated and requires various utilities to put all elements in place.

There is thus a need for facilitating assembly of medicament delivery devices.

This need is met by a device according to the present disclosure.

### SUMMARY

Reference should now be made to the appended claims.

The invention refers to a medicament delivery device, the medicament delivery device having a proximal end pointing towards a dose delivery site during use of the medicament delivery device, and a distal end opposite the proximal end, the medicament delivery device comprising:
a housing having a longitudinal axis,
a medicament delivery member cover axially moveable with respect to the housing,
wherein the housing comprises an inner cylindrical portion, the cylindrical portion comprising a flange extending in the radial outward direction with respect to the longitudinal axis,
wherein the medicament delivery member cover comprises at least one medicament delivery member cover arm extending in a distal direction away from a medicament delivery site, wherein the medicament delivery member cover arm comprises a flexible locking arm extending in a radial inward direction, and
wherein the flexible locking arm is configured to abut the flange and to block proximal movement of the medicament delivery member cover towards the medicament delivery site.

In a non-limiting embodiment, the flexible locking arm comprises a relaxed state and a compressed state, and in the relaxed state, the flexible locking arm extends in the proximal direction and the radial inward direction, and in the compressed state during assembly, the flexible locking arm substantially extends only in the proximal direction.

In a non-limiting embodiment, during assembly, the medicament delivery member cover is inserted into the housing in the distal direction, and the flexible locking arm is configured to bend radially outwards when passing the flange and to flex back radially inwards when a distal end of the flange is passed.

In a non-limiting embodiment, the housing comprises a connecting portion connecting an inner circumferential surface of the housing with an outer circumferential surface of the inner cylindrical portion.

In a non-limiting embodiment, the housing comprises a window, wherein the connecting portion surrounds the window.

In a non-limiting embodiment, the flange forms a circular section on the outer circumferential surface of the inner cylindrical portion.

In a non-limiting embodiment, the flange circumferentially extends around the inner cylindrical portion.

In a non-limiting embodiment, the medicament delivery member cover comprises two medicament delivery member cover arms.

The invention is specified by the independent claim. Preferred embodiments are defined in the dependent claims. In the following description, although numerous features may be designated as optional, it is nevertheless acknowledged that all features comprised in the independent claims are not to be read as optional.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIGS. 1A and 1B show a housing according to an embodiment of the disclosure.

FIG. 1C shows a medicament delivery member cover according to an embodiment of the present disclosure.

FIG. 2 shows a perspective view of the housing according to FIGS. 1A and 1B.

FIGS. 3A and 3B show sectional views of the housing according to FIGS. 1A and 1B.

FIG. 4 shows a sectional view of an assembled medicament delivery device according to an embodiment of the present disclosure.

FIG. 5 shows an exploded view of a medicament delivery device according to an embodiment of the present disclosure.

### DETAILED DESCRIPTION

The assembly mechanism according to the present disclosure can be employed for various medicament delivery devices. Thus, only the parts relevant for carrying out the disclosure will be described in detail.

In the figures, elements not essential for carrying out the invention have been omitted. In particular, not all figures show a complete outer housing, biasing members, a medicament container, cap, etc. in order not to render the depictions overly complicated and not to blur the scope of the disclosure.

In the following, the assembled state refers to a state in which the device is delivered to the patient, i.e., before activation and medicament delivery. In the assembled state, the plunger rod is in a locked position and not able to rotate or move in the proximal direction. In the activation state, the medicament delivery member cover is moved in the distal direction along the longitudinal axis from an extended position to a retracted position. In the activated state, the plunger rod is free to rotate and move in the proximal direction for medicament delivery. The plunger rod may thus be displaceable along the axial direction.

The medicament delivery member cover can be moved from the extended to the retracted position. Therefore, the medicament delivery member cover can be displaced in the axial direction. In the extended position, the medicament delivery member cover extends in a proximal direction from the housing. Thereby, a medicament delivery member, e.g., a needle, may be covered by the medicament delivery member cover. By pushing the medicament delivery member cover in the distal direction, e.g., by pushing the device onto a medicament delivery site, the medicament delivery member cover is moved to the retracted position for activating medicament delivery. After delivery, the medicament delivery member cover may be moved back to the extended position to cover the medicament delivery member. The medicament delivery member cover may also be locked in the extended position after medicament delivery to and prevent injuries of the patient.

In the following, the term engagement encompasses any kind of interaction between the named features such as abutment, clamping, locking, biasing, flexing, etc.

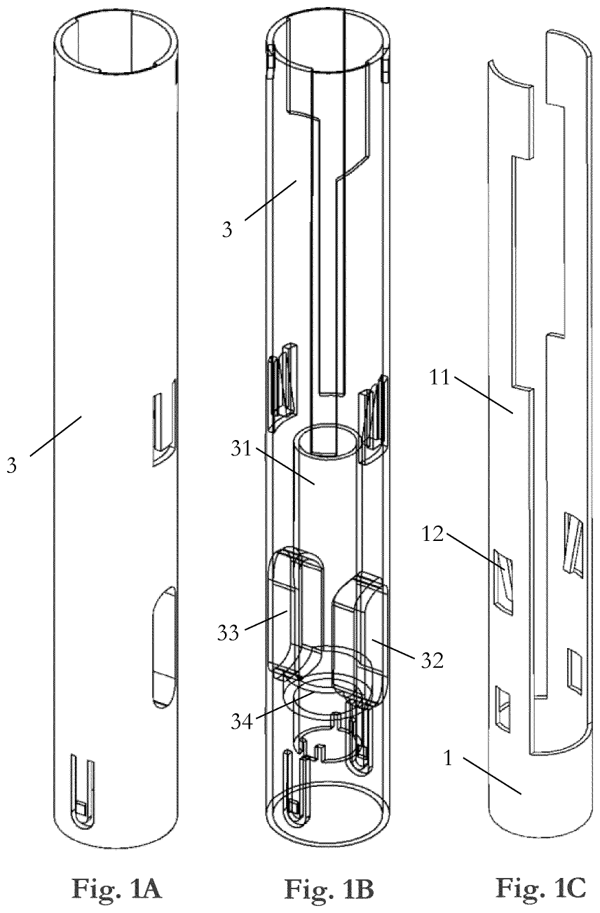

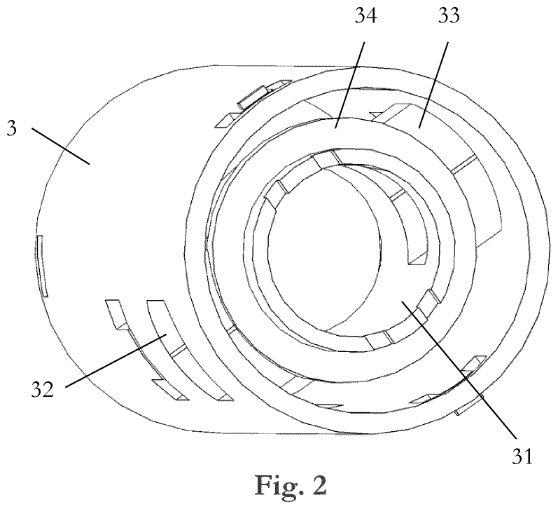

The present disclosure relates to a medicament delivery device. With reference to FIG. 1A, the medicament delivery device comprises a housing 3 having a longitudinal axis. The housing 3 may thus substantially be formed as an elongated tubular member.

FIG. 1B shows the housing 3 of FIG. 1A in a transparent view. The housing 3 comprises an inner cylindrical portion 31 comprising a flange 34 (collar, shroud) extending in the radial outward direction. That is, the flange 34 may be provided on an outer circumferential surface of the inner cylindrical portion 31 and extend therefrom in the radial outward direction. The inner cylindrical portion 31 may receive a syringe 5 (not shown) comprising a medicament. The flange 34 may be provided at or towards a proximal end of the inner cylindrical portion 31. The flange 34 may form a circular section on the inner cylindrical portion 31. Alternatively, the flange 34 may extend around the whole circumference of the inner cylindrical portion 31.

The inner cylindrical portion 31 may be connected to an inner circumferential surface of the housing 3 via a connection portion 33. The connection portion 33 may be formed of one or more parts (e.g., bars, thwarts). The housing 3 may further comprise a window 32 for allowing a patient to view the contents of the syringe, wherein the window 32 is formed as a cut-out in the housing 3. The connection portion 33 may surround the window 32 and may thus form a radially extending wall connecting the inner circumferential surface of the housing 3 to the inner cylindrical portion 31.

The medicament delivery device further comprises a medicament delivery member cover 1 axially moveable with respect to the housing 3. In particular, the medicament delivery member cover 1 is axially moveable within the housing 3.

The medicament delivery member cover 1 comprises at least one medicament delivery member cover arm 11 extending in a distal direction away from a medicament delivery site. The medicament delivery member cover arm 11 comprises a flexible locking arm 12 extending in a radial inward direction.

The flexible locking arm 12 is configured to abut the flange 34 and to block proximal movement of the medicament delivery member cover 1 towards the medicament delivery site.

In the figures, two medicament delivery member cover arms 11 are shown which are identical in construction. The number, however, is not to be understood as limiting.

FIG. 2 shows a perspective view of the housing 3. In particular, FIG. 2 shows the housing 3 when viewed from the proximal direction, i.e., from the medicament delivery site.

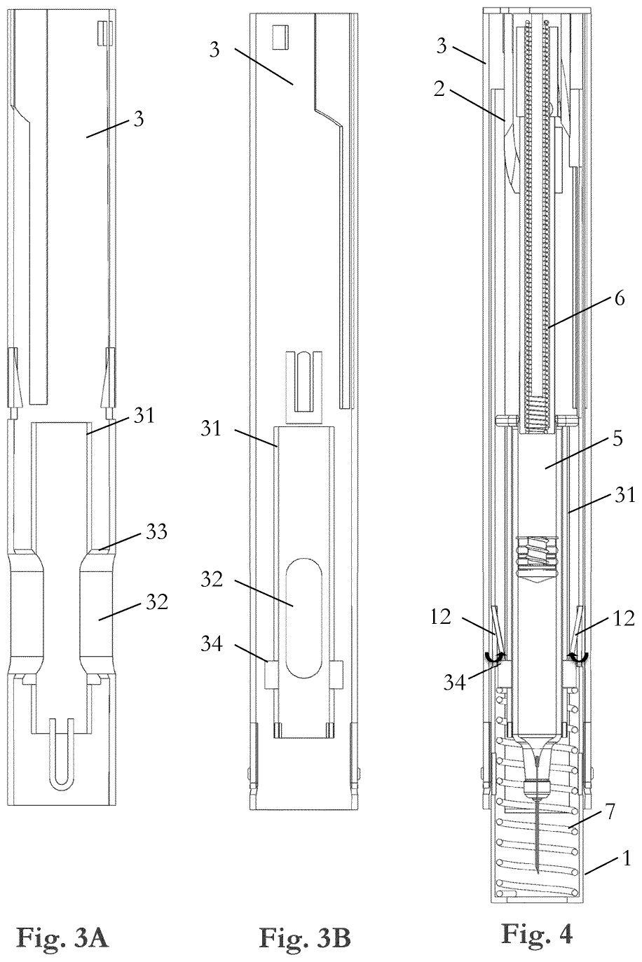

FIG. 3A is a sectional view of the housing 3 along the longitudinal axis and FIG. 3B shows the housing 3 of FIG. 3A rotated by 90 degrees around the longitudinal axis.

FIG. 4 is a sectional view of a medicament delivery device in the assembled state wherein the view corresponds to the orientation of FIG. 3B.

The flexible locking arms 12 may comprise a relaxed state and a compressed state. In the relaxed state, the flexible locking arms 12 extend in the proximal direction and the radial inward direction as shown in FIGS. 1C and 4. The relaxed state may refer to a state after manufacturing in which the locking arms 12 are moulded so as to extend inward as shown in the figures. In other words, the locking arms 12 are pre-tensioned during moulding.

During the assembly process, the medicament delivery member cover 1 is inserted into the housing 3 in the distal direction, i.e., through the proximal opening of the housing 3 (from the front). The flexible locking arms 12 are configured to bend/flex radially outwards when passing the flange 34 into a compressed state. In the compressed state during assembly, the flexible locking arms 12 substantially extend in the proximal direction. In particular, the flexible locking arms 12 may substantially extend only in the proximal direction. In other words, the flexible locking arms 12 may substantially align with the housing 3 in the axial direction. The flexible locking arms 12 are further configured to flex back radially inwards when a distal end of the flange 34 is passed, i.e., when the medicament delivery member cover 1 has been pushed far enough in the distal direction. The flange 34 thus serves as an attachment point.

This flexible arrangement of the locking arms 12 allows for minimum introduction/assembly force and does not require further tools. After passing the flange 34, the flexible locking arms 12 automatically flex back outwards and thus secure the medicament delivery member cover 1 in the housing 3 by blocking proximal movement through the abutment on the flange 34. This is indicated by the arrows in FIG. 4.

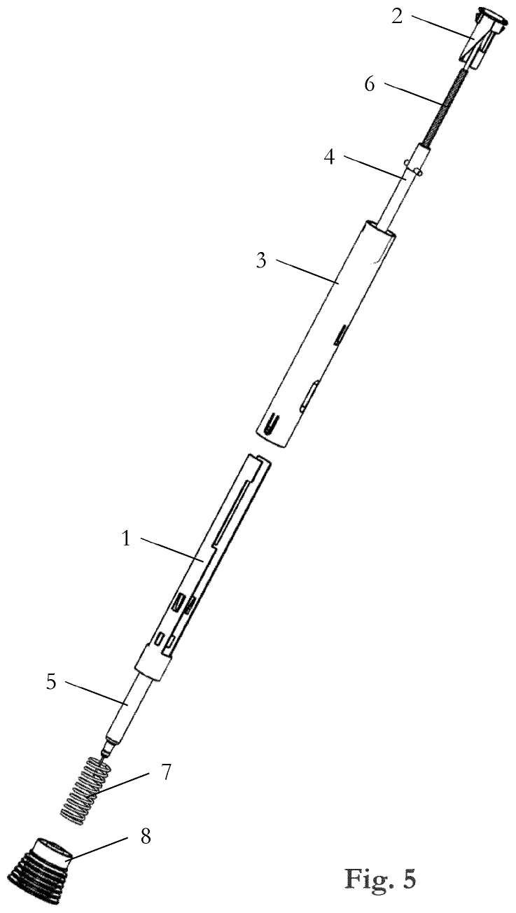

FIG. 5 shows an exploded view of a medicament delivery device as described above comprising a cap 8, a medicament delivery member cover biasing member 7 (for instance a coil spring), a syringe 5, a medicament delivery member cover 1, a housing 3, a plunger rod 4, a drive biasing member 6 (for instance a coil spring) and a rear housing 2.

In the present disclosure, improved mechanisms for assembly of a medicament delivery device have been presented. In particular, the flexible arms allow a simple and secure assembly of the medicament delivery member cover without the necessity of complex structures on the medicament delivery member cover arms or complex tools for assembly of the device.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

### REFERENCE SIGNS

| | |
|---|---|
| 1 | Medicament delivery member cover/needle cover |
| 11 | Medicament delivery member cover arm/needle cover arm |
| 12 | Locking arm |
| 2 | Rear housing/rear cap |
| 3 | Housing |
| 31 | Inner cylindrical portion |
| 32 | Window |
| 33 | Connecting portion |
| 34 | Flange |
| 4 | (Rotatable) Plunger rod |
| 41 | Plunger rod boss |
| 5 | Syringe |
| 6 | Drive biasing member |
| 7 | Medicament delivery member cover biasing member/needle |
| | cover biasing member |
| 8 | Cap |

1-8. (canceled)

9. A medicament delivery device, the medicament delivery device having a proximal end pointing towards a dose delivery site during use of the medicament delivery device, and a distal end opposite the proximal end, the medicament delivery device comprising:
a housing having a longitudinal axis; and
a medicament delivery member cover axially moveable with respect to the housing,
wherein the housing comprises an inner cylindrical portion, the cylindrical portion comprising a flange extending in the radial outward direction with respect to the longitudinal axis,
wherein the medicament delivery member cover comprises at least one medicament delivery member cover arm extending in a distal direction away from a medicament delivery site, wherein the medicament delivery member cover arm comprises a flexible locking arm extending in a radial inward direction, and
wherein the flexible locking arm is configured to abut the flange and to block proximal movement of the medicament delivery member cover towards the medicament delivery site.

10. Medicament delivery device according to claim 9, wherein the flexible locking arm comprises a relaxed state and a compressed state, wherein in the relaxed state, the flexible locking arm extends in the proximal direction and the radial inward direction, and in the compressed state during assembly, the flexible locking arm substantially extends only in the proximal direction.

11. Medicament delivery device according to claim 9, wherein during assembly, the medicament delivery member cover is inserted into the housing in the distal direction, and the flexible locking arm is configured to bend radially outwards when passing the flange and to flex back radially inwards when a distal end of the flange is passed.

12. Medicament delivery device according to claim 9, wherein the housing comprises a connecting portion connecting an inner circumferential surface of the housing with an outer circumferential surface of the inner cylindrical portion.

13. Medicament delivery device according to claim 12, wherein the housing comprises a window, wherein the connecting portion surrounds the window.

14. Medicament delivery device according to claim 9, wherein the flange forms a circular section on the outer circumferential surface of the inner cylindrical portion.

15. Medicament delivery device according to claim 9, wherein the flange circumferentially extends around the inner cylindrical portion.

16. Medicament delivery device according to claim 9, wherein the medicament delivery member cover comprises two medicament delivery member cover arms.
